(19) United States
(12) Patent Application Publication
(10) Pub. No.: US 2026/0347796 A1
(43) Pub. Date: Nov. 5, 2026

(54) HELPER PLASMID FOR HIGH-YIELD PRODUCTION OF RECOMBINANT ADENO-ASSOCIATED VIRUS, AND USE THEREOF

(71) Applicant: PACKGENE BIOTECH CO., LTD., Guangzhou (CN)

(72) Inventors: Huapeng Li, Guangzhou (CN); Danqing Xiao, Guangzhou (CN); Ye Bu, Guangzhou (CN)

(21) Appl. No.: 19/701,727

(22) Filed: Jun. 8, 2026

Related U.S. Application Data
(63) Continuation of application No. PCT/CN2024/130741, filed on Nov. 8, 2024.

(30) Foreign Application Priority Data
Dec. 13, 2023 (CN) 202311709062.5

(51) Int. Cl. C12N 7/02

(52) U.S. Cl. CPC C12N 7/025; C12N 2750/14152; C12N 2800/10; C12N 2830/34

(57) ABSTRACT

Provided are a helper plasmid for high-yield production of a recombinant adeno-associated virus (rAAV), and a use thereof. The helper plasmid includes a backbone plasmid and a recombination sequence. The recombination sequence includes: a Rep protein-encoding sequence, a Cap protein-encoding sequence, an E2A sequence, an E4 sequence, a VA RNA-encoding DNA sequence, and at least one promoter sequence. The at least one promoter sequence is located downstream of the Rep protein-encoding sequence and the Cap protein-encoding sequence. After being transfected into cells, the helper plasmid can significantly increase the adeno-associated virus (AAV) yield per cell. The helper plasmid can be used in large-scale AAV production.

### CROSS-REFERENCE TO RELATED APPLICATIONS

The present application is a Continuation Application of PCT application No. PCT/CN2024/130741 filed on Nov. 8, 2024, which claims the benefit of Chinese Patent Application No. 202311709062.5 filed on Dec. 13, 2023. The contents of the above-identified applications are hereby incorporated by reference in their entirety.

### REFERENCE TO SEQUENCE LISTING

This application includes a Sequence Listing filed electronically as an XML file named “US2601077H-PCT_SL.xml”, created on May 7, 2026, with a size of 82,340 bytes. The Sequence Listing is incorporated herein by reference.

### TECHNICAL FIELD

The present application relates to the technical field of genetic engineering, and specifically relates to a helper plasmid for high-yield production of a recombinant adeno-associated virus (rAAV), and a use thereof.

### BACKGROUND

In recent years, rAAV-based gene therapy has garnered widespread attention due to advantages such as low pathogenicity and low immunogenicity. Consequently, rAAV has become a highly favored delivery vector for gene therapy. rAAV-based vectors can target both dividing and non-dividing cells, including those in the retina, liver, heart, muscle, and central nervous system (CNS), so as to achieve long-term expression of therapeutic genes. More than 200 clinical trials have been conducted for the treatment of various diseases, such as hemophilia, Parkinson's disease, wet age-related macular degeneration, mucopolysaccharidosis (MPS), and Batten disease. Despite the success of rAAV-based gene therapy in clinical trials, high production costs remain a bottleneck in the gene therapy field due to low yields and high dose requirements of some disease indications, which limits the access of patients to these promising products. As a result, the demand for reducing adeno-associated virus (AAV) production costs and establishing flexible, Good Manufacturing Practice (GMP)-compliant AAV production systems has been intensifying.

Transient transfection of human embryonic kidney (HEK) 293 cells with plasmids is a common strategy for the preparation of AAV vectors, offering major advantages including short production cycle, easy implementation, and high flexibility. The triple-plasmid transfection method is currently the most common rAAV production method. This triple-plasmid transfection method involves co-transfection of HEK293 cells with three plasmids: an AAV genome plasmid including a gene of interest (GOI) expression cassette flanked by inverted terminal repeats (ITRs) at two termini, a packaging plasmid providing coding sequences for AAV Rep and Cap proteins, and a helper plasmid harboring adenoviral E2A, E4, and VA RNA genes.

With the rapid growth in demand for rAAV vectors in gene therapy, rAAV production processes have gradually evolved from triple-plasmid AAV packaging systems to efficient and scalable two-plasmid systems. The core concept for the two-plasmid systems is to integrate two of rep/cap, a GOI expression cassette, and helper, thereby reducing transfection complexity and optimizing the expression ratio among various functional modules to some extent. Currently, representative two-plasmid systems include the “rep/cap+helper integrated” system of pQT (derived from pDG), the “GOI+helper integrated” system of pLV, and the “GOI+rep/cap integrated” system of pOXB. The pLV system integrates GOI and adenoviral helper elements into a single plasmid, resulting in the coupling of helper expression with GOI expression. Consequently, the pLV system is not conducive to optimizing the optimal ratio of rep/cap to helper, and thus may limit overall production efficiency and consistency. The pOXB system integrates GOI with rep/cap to achieve a favorable expression ratio and a high full capsid proportion. However, the significantly increased size of the plasmid aggravates the construction complexity. The integration of rep/cap and the GOI in a single vector theoretically increases the risk of unintended recombination or aberrant packaging events. Both pLV and pOXB systems still exhibit some deficiencies in terms of expression regulation flexibility, construction complexity, and potential process risks. In contrast, the pQT system offers advantages such as simple structure, independent modules, and excellent replaceability (suitable for rapid switching among different serotype capsids) while maintaining high yields, and is more in line with requirements of process development and GMP manufacturing. Therefore, modification based on the pQT system holds significant potential, and is expected to balance yield, quality, and process stability without substantially increasing system complexity, which is an important direction for the optimization of current two-plasmid AAV production systems.

### SUMMARY

An objective of the present application is to overcome deficiencies of the prior art, and provide a helper plasmid for high-yield production of rAAV, and a use thereof.

In order to achieve the above objective, the present application adopts the following

### Technical Solutions

In a first aspect, the present application provides a helper plasmid for producing rAAV, including: a backbone plasmid and a recombination sequence, where the recombination sequence includes: a Rep protein-encoding sequence, a Cap protein-encoding sequence, an E2A sequence, an E4 sequence, a VA RNA-encoding DNA sequence, and at least one promoter sequence; and the at least one promoter sequence is located downstream of the Rep protein-encoding sequence and the Cap protein-encoding sequence.

Since the promoter is located downstream of the Rep protein-encoding sequence and the Cap protein-encoding sequence, the helper plasmid of the present application can lead to an approximately 4.2-fold to 11.2-fold higher AAV yield than a pRCHelper helper vector driven by a single promoter after being transfected into cells. Consequently, the helper plasmid of the present application can significantly enhance the AAV yield per cell.

In some such helper plasmids, the recombination sequence further includes at least one DA′ sequence.

In some such helper plasmids, the at least one DA′ sequence is located downstream of the Rep protein-encoding sequence and the Cap protein-encoding sequence.

In some such helper plasmids, the recombination sequence includes, in a 5′ to 3′ direction, any one selected from the group consisting of the following combinations:
i. the Rep protein-encoding sequence, the Cap protein-encoding sequence, the E2A sequence, the E4 sequence, the VA RNA-encoding DNA sequence, and a first promoter sequence;
ii. the Rep protein-encoding sequence, the Cap protein-encoding sequence, the E2A sequence, the E4 sequence, the VA RNA-encoding DNA sequence, the DA′ sequence, and the first promoter sequence;
iii. the E2A sequence, the E4 sequence, the VA RNA-encoding DNA sequence, the Rep protein-encoding sequence, the Cap protein-encoding sequence, the DA′ sequence, and the first promoter sequence;
iv. the Rep protein-encoding sequence, the Cap protein-encoding sequence, the first promoter sequence, the E2A sequence, the E4 sequence, and the VA RNA-encoding DNA sequence;
v. the Rep protein-encoding sequence, the Cap protein-encoding sequence, the DA′ sequence, the first promoter sequence, the E2A sequence, the E4 sequence, and the VA RNA-encoding DNA sequence;
vi. the Rep protein-encoding sequence, the Cap protein-encoding sequence, the first promoter sequence, the E2A sequence, the E4 sequence, the VA RNA-encoding DNA sequence, and the DA′ sequence;
vii. the Rep protein-encoding sequence, the Cap protein-encoding sequence, the DA′ sequence, the E2A sequence, the E4 sequence, the VA RNA-encoding DNA sequence, and the first promoter sequence;
viii. the Rep protein-encoding sequence, the Cap protein-encoding sequence, the DA′ sequence, the E2A sequence, the E4 sequence, the VA RNA-encoding DNA sequence, the DA′ sequence, and the first promoter sequence; and
ix. the Rep protein-encoding sequence, the Cap protein-encoding sequence, the DA′ sequence, the first promoter sequence, the E2A sequence, the E4 sequence, the VA RNA-encoding DNA sequence, the DA′ sequence, and a second promoter sequence.

In some examples of the helper plasmid, the recombination sequence includes, in the 5′ to 3′ direction, sequences of the combination i. That is, the recombination sequence includes the Rep protein-encoding sequence, the Cap protein-encoding sequence, the E2A sequence, the E4 sequence, the VA RNA-encoding DNA sequence, and the first promoter sequence sequentially. Optionally, there is no spacer sequence among the Rep protein-encoding sequence, the Cap protein-encoding sequence, the E2A sequence, the E4 sequence, the VA RNA-encoding DNA sequence, and the first promoter sequence. Optionally, there is a spacer sequence between any adjacent sequences of the Rep protein-encoding sequence, the Cap protein-encoding sequence, the E2A sequence, the E4 sequence, the VA RNA-encoding DNA sequence, and the first promoter sequence. Optionally, there is no spacer sequence between the Rep protein-encoding sequence and the Cap protein-encoding sequence (for example, as shown in A to J of FIG. 2), there is no spacer sequence among the E2A sequence, the E4 sequence, and the VA RNA-encoding DNA sequence (for example, as shown in A to J of FIG. 2), there is a spacer sequence between the Cap protein-encoding sequence and the E2A sequence (which is a non-coding nucleotide sequence that is typically removed or not involved in translation during gene expression), and there is a spacer sequence between the VA RNA-encoding DNA sequence and the first promoter sequence.

In some examples of the helper plasmid, the recombination sequence includes, in the 5′ to 3′ direction, sequences of the combination ii. That is, the recombination sequence includes the Rep protein-encoding sequence, the Cap protein-encoding sequence, the E2A sequence, the E4 sequence, the VA RNA-encoding DNA sequence, the DA′ sequence, and the first promoter sequence sequentially. Optionally, there is no spacer sequence among the Rep protein-encoding sequence, the Cap protein-encoding sequence, the E2A sequence, the E4 sequence, the VA RNA-encoding DNA sequence, the DA′ sequence, and the first promoter sequence. Optionally, there is a spacer sequence between any adjacent sequences of the Rep protein-encoding sequence, the Cap protein-encoding sequence, the E2A sequence, the E4 sequence, the VA RNA-encoding DNA sequence, and the first promoter sequence. Optionally, there is no spacer sequence between the Rep protein-encoding sequence and the Cap protein-encoding sequence (for example, as shown in A to J of FIG. 2), there is no spacer sequence among the E2A sequence, the E4 sequence, and the VA RNA-encoding DNA sequence (for example, as shown in A to J of FIG. 2), and there is a spacer sequence between the Cap protein-encoding sequence and the E2A sequence, between the VA RNA-encoding DNA sequence and the DA′ sequence, and between the DA′ sequence and the first promoter sequence.

Similarly, in some examples of the helper plasmid, the recombination sequence includes, in the 5′ to 3′ direction, sequences of the combination iii, iv, v, vi, vii, or viii. Optionally, there may be a spacer sequence or no spacer sequence between any adjacent sequences of the sequences of the combination, or there is a spacer sequence between some adjacent sequences but no spacer sequence between other adjacent sequences.

In some examples of the helper plasmid, the recombination sequence includes, in the 5′ to 3′ direction, sequences of the combination ix. That is, the recombination sequence includes the Rep protein-encoding sequence, the Cap protein-encoding sequence, the DA′ sequence, the first promoter sequence, the E2A sequence, the E4 sequence, the VA RNA-encoding DNA sequence, the DA′ sequence, and the second promoter sequence sequentially. In some examples of the helper plasmid, the first promoter sequence and the second promoter sequence are the same. In some examples of the helper plasmid, the first promoter sequence and the second promoter sequence are different. Optionally, there is no spacer sequence among the Rep protein-encoding sequence, the Cap protein-encoding sequence, the DA′ sequence, the first promoter sequence, the E2A sequence, the E4 sequence, the VA RNA-encoding DNA sequence, the DA′ sequence, and the second promoter sequence. Optionally, there is a spacer sequence between any adjacent sequences of the Rep protein-encoding sequence, the Cap protein-encoding sequence, the DA′ sequence, the first promoter sequence, the E2A sequence, the E4 sequence, the VA RNA-encoding DNA sequence, the DA′ sequence, and the second promoter sequence. Optionally, there is no spacer sequence between the Rep protein-encoding sequence and the Cap protein-encoding sequence (for example, as shown in A to J of FIG. 2), there is no spacer sequence among the E2A sequence, the E4 sequence, and the VA RNA-encoding DNA sequence (for example, as shown in A to J of FIG. 2), and there is a spacer sequence between the Cap protein-encoding sequence and the DA′ sequence, between the DA′ sequence and the first promoter sequence, between the first promoter sequence and the E2A sequence, between the VA RNA-encoding DNA sequence and the DA′ sequence, and between the DA′ sequence and the second promoter sequence.

In some such helper plasmids, the at least one promoter sequence is a P5 promoter sequence, and/or the first promoter sequence and/or the second promoter sequence are/is a P5 promoter sequence. Optionally, the P5 promoter sequence is set forth in SEQ ID NO: 2.

In some such helper plasmids, the DA′ sequence is set forth in SEQ ID NO: 3.

In some such helper plasmids, the Rep protein-encoding sequence is derived from AAV serotype 2 and the Cap protein-encoding sequence is derived from AAV serotype 9. Optionally, the Rep protein-encoding sequence is set forth in SEQ ID NO: 4 and/or the Cep protein-encoding sequence is set forth in SEQ ID NO: 5.

In some such helper plasmids, the E2A sequence, the E4 sequence, and the VA RNA-encoding DNA sequence are derived from an adenovirus. Optionally, a nucleotide sequence of the E2A sequence is set forth in SEQ ID NO: 6, a nucleotide sequence of the E4 sequence is set forth in SEQ ID NO: 7, and the VA RNA-encoding DNA sequence is set forth in SEQ ID NO: 8.

In some such helper plasmids, the first promoter sequence is set forth in SEQ ID NO: 2, the second promoter sequence is set forth in SEQ ID NO: 2, the DA′ sequence is set forth in SEQ ID NO: 3, the Rep protein-encoding sequence is set forth in SEQ ID NO: 4, the Cap protein-encoding sequence is set forth in SEQ ID NO: 5, a nucleotide sequence of the E2A sequence is set forth in SEQ ID NO: 6, a nucleotide sequence of the E4 sequence is set forth in SEQ ID NO: 7, and the VA RNA-encoding DNA sequence is set forth in SEQ ID NO: 8.

In a second aspect, the present application provides a plasmid combination for producing rAAV, including the helper plasmid and an AAV plasmid.

In some such plasmid combinations, the AAV plasmid is an AAV plasmid including CAG promoter-driven fluorescent protein DNA. In some such plasmid combinations, the AAV plasmid is a pAAV.CAG.EGFP vector.

In a third aspect, the present application provides a system for producing rAAV, where the system is prepared by transforming a host cell with the plasmid combination.

In some such systems, the host cell is a HeLa, HEK293, or insect Sf9 cell.

In a fourth aspect, the present application provides a method for producing rAAV, including: producing the rAAV using the system.

In a fifth aspect, the present application provides a use of the helper plasmid, the plasmid combination, or the system in AAV production.

Compared with the prior art, the present application has the following beneficial effects:

The helper plasmid provided in the present application can increase the rAAV yield. The helper plasmid includes a backbone plasmid and a recombination sequence. The recombination sequence includes: a Rep protein-encoding sequence, a Cap protein-encoding sequence, an E2A sequence, an E4 sequence, a VA RNA-encoding DNA sequence, and at least one promoter sequence. The at least one promoter sequence is located downstream of the Rep protein-encoding sequence and the Cap protein-encoding sequence. Since the promoter is located downstream of the Rep protein-encoding sequence and the Cap protein-encoding sequence, the helper plasmid of the present application can lead to an approximately 4.2-fold to 11.2-fold higher AAV yield than a single-promoter-driven pRCHelper helper vector (i.e., a pRCHelper helper vector driven by a single promoter) after being transfected into cells. Consequently, the helper plasmid of the present application can significantly enhance the AAV yield per cell. The helper plasmid can be used in large-scale AAV production.

### BRIEF DESCRIPTION OF THE DRAWINGS

FIG. 1 is a schematic diagram of structural elements of a plasmid backbone in Example 1;

FIG. 2 is a schematic diagram of an experimental design of candidate helper plasmids for studying functions of P5 (i.e., P5 promoter) and DA′ in an example;

FIG. 3 shows comparison of viral titers in cell lysates of various plasmid groups with a viral titer of a control vector, as detected in an example; and

FIG. 4 shows titer statistics for AAVs produced by two-plasmid and three-plasmid systems.

### DETAILED DESCRIPTION OF THE EMBODIMENTS

To well explain the objective, technical solutions, and advantages of the present application, the present application will be further explained below with reference to specific examples. It should be understood by those skilled in the art that the specific examples described herein are merely intended to explain the present application, rather than to limit the present application.

In the following examples, unless otherwise specified, the experimental methods adopted are conventional, and the materials and reagents adopted are commercially available.

### Example 1: Construction of Helper Plasmids pRCHelper

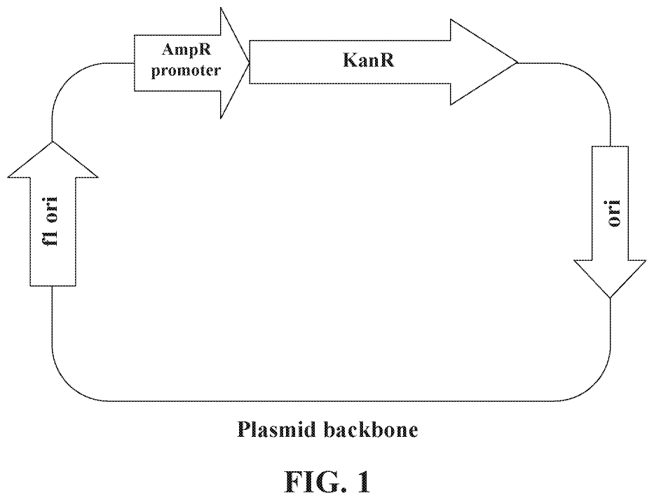

Different combinations of arrangement positions of various elements including DA′, P5 promoter, Rep, Cap, E2A, E4, and VA RNA were designed. Accordingly, different candidate helper plasmids were constructed based on a backbone vector by a conventional molecular cloning method (FIG. 1 and Table 1). Virus production titers of these helper vectors in a two-plasmid transfection system were then compared in parallel.

TABLE 1
Information of plasmids A to J

| Group | Plasmid information |
|---|---|
| A | P5 + RepCap + E2A/E4/VA RNA |
| B | RepCap + E2A/E4/VA RNA + P5 |
| C | RepCap + E2A/E4/VA RNA + DA′ + P5 |
| D | E2A/E4/VA RNA + RepCap + DA′ + P5 |
| E | RepCap + P5 + E2A/E4/VA RNA |
| F | RepCap + DA′ + P5 + E2A/E4/VA RNA |
| G | RepCap + P5 + E2A/E4/VA RNA + DA′ |
| H | RepCap + DA′ + E2A/E4/VA RNA + P5 |
| I | RepCap + DA′ + E2A/E4/VA RNA + DA′ + P5 |
| J | RepCap + DA′ + P5 + E2A/E4/VA RNA + DA′ + P5 |

Notes:
In Table 1 and throughout the present application, the “/” between elements, for example, “E2A” and “E4” does not indicate an “or” relationship between E2A and E4, but means that E2A and E4 are adjacent to each other.

The plasmid backbone sequence (as shown in FIG. 1) is set forth in SEQ ID NO: 1. The P5 promoter sequence is set forth in SEQ ID NO: 2. The DA′ sequence is set forth in SEQ ID NO: 3. The Rep (Rep protein-encoding sequence) is derived from AAV serotype 2, and has a sequence set forth in SEQ ID NO: 4. The Cap (Cap protein-encoding sequence) is derived from AAV serotype 9, and has a sequence set forth in SEQ ID NO: 5. The E2A, E4, and VA RNA (VA RNA-encoding DNA sequence) are derived from an adenovirus. The E2A sequence is set forth in SEQ ID NO: 6. The E4 sequence is set forth in SEQ ID NO: 7. The VA RNA-encoding DNA sequence is set forth in SEQ ID NO: 8.

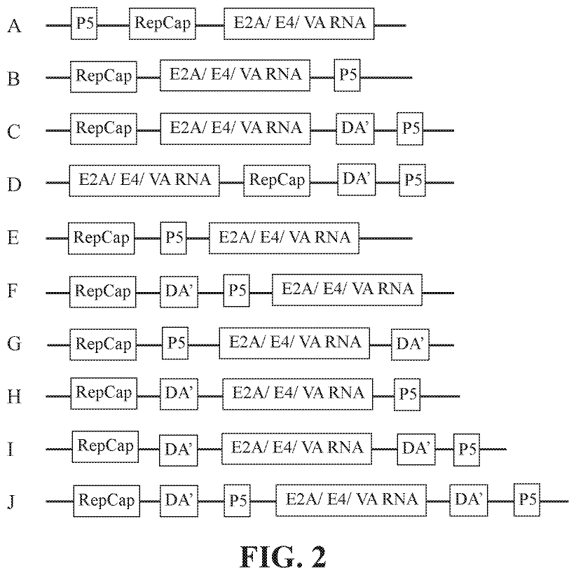

The construction process for the helper plasmids A to J in FIG. 2 was as follows:

(1) With a plasmid including a RepCap gene (which was constructed from plasmid #130878 of ADDGENE and GenBank sequence AY530579.1, with a vector sequence set forth in SEQ ID NO: 59) as a template, the RepCap gene sequence, the DA′ sequence, the P5 sequence, and a gene sequence including E2A, E4, and VA RNA (E2A/E4/VA RNA) were amplified separately.

A PCR system was shown in Table 2:

TABLE 2
Polymerase chain reaction (PCR) system

| Reaction component | Volume (μL) |
|---|---|
| 5 × Q5 Reaction Buffer | 10 |
| 10 mM dNTPs | 1 |
| Q5 Hot Start High-Fidelity DNA | 0.5 |
| Polymerase | |
| Forward primer | 2.5 |
| Reverse primer | 2.5 |
| Plasmid template | 1 |
| Deionized water | 32.5 |

Primer sequences used for constructing a plasmid A were as follows:

a forward primer 1:
(SEQ ID NO: 9)
ATGGCTGCCGATGGTTATC;
a reverse primer 1:
(SEQ ID NO: 10)
acgtaatccgtagatgtacctgg;
a forward primer 2:
(SEQ ID NO: 11)
aggtacatctacggattacgtAAGCCGAATTCTGCAGATATCC;
a reverse primer 2:
(SEQ ID NO: 12)
gtgacctctaatacaggacctAGCTCCCCCGATACCGTC;
a forward primer 3:
(SEQ ID NO: 13)
aggtcctgtattagaggtcacg;
and
a reverse primer 3:
(SEQ ID NO: 14)
ACCATCGGCAGCCATacctgatttaaatcatttattgttcaaagatg.

A Cap+E2A/E4/VA RNA fragment was amplified with the primer pair 1. A backbone fragment was amplified with the primer pair 2. A P5+Rep fragment was amplified with the primer pair 3.

Primer sequences used for constructing a plasmid B were as follows:

a forward primer 1:
(SEQ ID NO: 15)
aggtacatctacggattacgtTGCAGGACTAGAGGTCCTG;
a reverse primer 1:
(SEQ ID NO: 16)
AGCTCCCCCGATACCGTC;
a forward primer 2:
(SEQ ID NO: 17)
CGATCGAGGTCGACGGTATC;
and
a reverse primer 2:
(SEQ ID NO: 18)
acgtaatccgtagatgtacctgg.

A P5+backbone fragment was amplified with the primer pair 1. A RepCap+E2A/E4/VA RNA fragment was amplified with the primer pair 2.

Primer sequences used for constructing a plasmid C were as follows:

a forward primer 1:
(SEQ ID NO: 19)
CCCCCTCGATCGAGGATGCCGGGGTTTTACGAGAT;
a reverse primer 1:
(SEQ ID NO: 20)
CCTCCCACCAGATCACCATC;
a forward primer 2:
(SEQ ID NO: 21)
aggtacatctacggattacgtACCTGCAAGGAACCCCTAGT;
a reverse primer 2:
(SEQ ID NO: 22)
gagttgggtaccggatccGTTCAACTGAAACGAATCAACCG;
a forward primer 3:
(SEQ ID NO: 23)
ggatccggtacccaactcca;
and
a reverse primer 3:
(SEQ ID NO: 24)
acgtaatccgtagatgtacctgg.

A Rep fragment was amplified with the primer pair 1. A DA′+P5+backbone+RepCap fragment was amplified with the primer pair 2. An E2A/E4/VA RNA fragment was amplified with the primer pair 3.

Primer sequences used for constructing a plasmid D were as follows:

a forward primer 1:
(SEQ ID NO: 25)
aggtacatctacggattacgtCGATCGAGGTCGACGGTATC;
a reverse primer 1:
(SEQ ID NO: 26)
gagttgggtaccggatccAAAACCCTGGCGTTACCCAA;
a forward primer 2:
(SEQ ID NO: 27)
ggatccggtacccaactcca;
and
a reverse primer 2:
(SEQ ID NO: 28)
acgtaatccgtagatgtacctgg.

A RepCap+DA′+P5+backbone fragment was amplified with the primer pair 1. An E2A/E4/VA RNA fragment was amplified with the primer pair 2.

Primer sequences used for constructing a plasmid E were as follows:

a forward primer 1:
(SEQ ID NO: 29)
CGAAGGGCGAATTCGTTTGCAGGACTAGAGGTCCTGTATTAG;
a reverse primer 1:
(SEQ ID NO: 30)
gggtaaataatcacccgagagt;
a forward primer 2:
(SEQ ID NO: 31)
aggtacatctacggattacgtTTCCAGTCGGGAAACCTGTC;
a reverse primer 2:
(SEQ ID NO: 32)
gagttgggtaccggatccACTTTATGCTTCCGGCTCGT;
a forward primer 3:
(SEQ ID NO: 33)
ggatccggtacccaactcca;
and
a reverse primer 3:
(SEQ ID NO: 34)
acgtaatccgtagatgtacctgg.

A P5+partial E2A fragment was amplified with the primer pair 1. A backbone+RepCap fragment was amplified with the primer pair 2. An E2A/E4/VA RNA fragment was amplified with the primer pair 3.

Primer sequences used for constructing a plasmid F were as follows:

a forward primer 1:
(SEQ ID NO: 35)
aggtacatctacggattacgtTTCCAGTCGGGAAACCTGTC;
a reverse primer 1:
(SEQ ID NO: 36)
gagttgggtaccggatccACTTTATGCTTCCGGCTCGT;
a forward primer 2:
(SEQ ID NO: 37)
ggatccggtacccaactcca;
and
a reverse primer 2:
(SEQ ID NO: 38)
acgtaatccgtagatgtacctgg.

A backbone+RepCap+DA′+P5 fragment was amplified with the primer pair 1. An E2A/E4/VA RNA fragment was amplified with the primer pair 2.

Primer sequences used for constructing a plasmid G were as follows:

a forward primer 1:
(SEQ ID NO: 39)
ggatccggtacccaactcca;
a reverse primer 1:
(SEQ ID NO: 40)
GTCCTGCAGCCTCAGTGA;
a forward primer 2:
(SEQ ID NO: 41)
ACTGAGGCTGCAGGACGTGGAGCTCCAGCTTTTGTT;
a reverse primer 2:
(SEQ ID NO: 42)
GTTCAACTGAAACGAATCAACCG;
a forward primer 3:
(SEQ ID NO: 43)
CGGTTGATTCGTTTCAGTTGAACTGCAGGACTAGAGGTCCTG;
and
a reverse primer 3:
(SEQ ID NO: 44)
gagttgggtaccggatccGGATATCTGCAGAATTCGGCTT.

An E2A/E4/VA RNA fragment was amplified with the primer pair 1. A backbone+RepCap fragment was amplified with the primer pair 2. A P5 fragment was amplified with the primer pair 3.

Primer sequences used for constructing a plasmid H were as follows:

a forward primer 1:
(SEQ ID NO: 45)
aggtacatctacggattacgtGCAGGACTAGAGGTCCTGTATTAG;
a reverse primer 1:
(SEQ ID NO: 46)
gagttgggtaccggatccGCCTCAGTGAGCGAGC;
a forward primer 2:
(SEQ ID NO: 47)
ggatccggtacccaactcca;
and
a reverse primer 2:
(SEQ ID NO: 48)
acgtaatccgtagatgtacctgg.

A P5+backbone+RepCap+DA′ fragment was amplified with the primer pair 1. An E2A/E4/VA RNA fragment was amplified with the primer pair 2.

Primer sequences used for constructing a plasmid I were as follows:

a forward primer 1:
(SEQ ID NO: 49)
CGGTTGATTCGTTTCAGTTGAAC;
a reverse primer 1:
(SEQ ID NO: 50)
gagttgggtaccggatccGCCTCAGTGAGCGAGC;
a forward primer 2:
(SEQ ID NO: 51)
ggatccggtacccaactcca;
and
a reverse primer 2:
(SEQ ID NO: 52)
GTTCAACTGAAACGAATCAACCG.

A DA′ fragment was amplified with the primer pair 1. An E2A/E4/VA RNA+DA′+P5+backbone+RepCap fragment was amplified with the primer pair 2.

Primer sequences used for constructing a plasmid J were as follows:

a forward primer 1:
(SEQ ID NO: 53)
CGGTTGATTCGTTTCAGTTGAAC;
a reverse primer 1:
(SEQ ID NO: 54)
tggagttgggtaccggatccCTGCAGAATTCGGCTTGG;
a forward primer 2:
(SEQ ID NO: 55)
ggatccggtacccaactcca;
and
a reverse primer 2:
(SEQ ID NO: 56)
GTTCAACTGAAACGAATCAACCG.

A DA′+P5 fragment was amplified with the primer pair 1. An E2A/E4/VA RNA+DA′+P5+backbone+RepCap fragment was amplified with the primer pair 2.

PCR conditions were shown in Table 3:

TABLE 3
PCR conditions

| Reaction step | Reaction temperature | Reaction time | |
|---|---|---|---|
| Initial Denaturation | 98 °C | 30 | s |
| 35 Cycles | 98 °C | 10 | s |
| | 56 °C | 10 | s |
| | 72 °C | 30 | s/kb |
| Final Extension | 72 °C | 2 | min |
| Hold | 8 °C | | |

(2) Bands amplified by PCR were detected by agarose gel electrophoresis. Target fragments were recovered using a gel extraction kit.

(3) Multi-fragment ligation was conducted using a seamless cloning kit.

A seamless cloning reaction system was shown in Table 4:

TABLE 4
Seamless cloning reaction system

| Reaction component | Volume (μL) |
|---|---|
| 2 × Assembly Mix | 5 |
| Linearized vector | 1 |
| Inserts | n |
| Nuclease-free Water | to 10 |

Multi-fragment ligation was conducted at 50 °C for 1 h.

A ligation product was transformed into Escherichia coli, and transformed Escherichia coli was spread onto a kanamycin-resistant plate. The next day, colonies were picked and identified by colony PCR. Positive colonies were sent to Guangzhou Genewiz Biotechnology Co., Ltd. for sequencing. Desired correct plasmids were selected.

### Example 2: Production of AAV Through Two-Plasmid Transfection

(1) Approximately 3×10⁵ 293T cells (293T, derived from ATCC® CRL-3216™) were inoculated into each well of a 6-well plate with high-glucose Dulbecco's Modified Eagle Medium (DMEM) (glucose concentration: 4.5 g/L) including 10% (v/v) newborn calf serum and 1% (v/v) penicillin/streptomycin, and cultured in a 37 °C and 5% (v/v) CO₂ incubator for about 48 h. When a cell density reached approximately 60% to 70%, transfection was conducted.

(2) Each of the pRCHelper helper plasmids A to J constructed in Example 1 and the AAV plasmid including CAG promoter-driven fluorescent protein DNA, namely, the pAAV.CAG.EGFP vector (constructed from plasmid #61463 of ADDGENE and GenBank sequence MK225672.1, with a vector sequence set forth in SEQ ID NO: 60), were added at a ratio of 1 μg:0.5 μg to 0.5 mL of high-glucose DMEM. Then, 3 μL of polyethylenimine (PEI) (1 μg/μL) was added. A resulting mixture was vortexed for thorough mixing, allowed to stand at room temperature for 10 min, and then added to 1.5 mL of a transfection medium that was prepared from high-glucose DMEM and included 1% (v/v) newborn calf serum and 1% (v/v) penicillin/streptomycin. Vortexing was conducted for thorough mixing to produce a mixed transfection medium.

For a three-plasmid control group, a pRC plasmid (the packaging vector “RC+DA′+P5” from a preferred embodiment in U.S. Ser. No. 12/466,858B2), a pHelper plasmid (synthesized using the sequence set forth in GenBank: AF369965.1), and the AAV plasmid including CAG promoter-driven fluorescent protein DNA, namely, the pAAV.CAG.EGFP vector, were added at a ratio of 0.5 μg:0.5 μg:0.5 μg to 0.5 mL of high-glucose DMEM. Then, 3 μL of PEI (1 μg/μL) was added. A resulting mixture was vortexed for thorough mixing, allowed to stand at room temperature for 10 min, and then added to 1.5 mL of a transfection medium. Vortexing was conducted for thorough mixing to produce a mixed transfection medium.

The original medium in the 6-well plate was removed, and the mixed transfection medium was added. The plate was then returned to a 37 °C incubator (5% (v/v) CO₂) and further incubated.

(3) After 72 h of culture, 150 μL of a cell lysis buffer (Gibco™, A50520) was added. A resulting mixture was collected into a 2 mL centrifuge tube, subjected to lysis on a shaker at 37 °C and 250 rpm for 1 h, and centrifuged at 10,000 g for 10 min to produce a supernatant, which was a crude AAV extract.

### Example 3: Determination of AAV Titers

An AAV titer produced after two-plasmid transfection in each group of Example 2 was determined. A specific determination method was as follows:

Sequences of ITRs of all AAV vectors were
specifically detected using primers
FWD ITR
(5′-GGAACCCCTAGTGATGGAGTT, SEQ ID NO: 57)
and
REV ITR
(5′-CGGCCTCAGTGAGCGA, SEQ ID NO: 58).

### (1) DNase I Digestion of Samples

Components of a reaction system were shown in Table 5:

TABLE 5
Reaction system

| Reagent | Volume | |
|---|---|---|
| Nuclease-free Water | 15.8 | μL |
| 10 × DNase Buffer | 2 | μL |
| Dnase I, RNase free (70 U/μL) | 0.2 | μl |
| Sample | 2 | μL |

5 μL of a sample was taken and diluted 20-fold. A corresponding number of PCR tubes were taken, and 18 μL of a digestion solution was added to each tube. Then, 2 μL of a diluted sample and 2 μL of a plasmid standard (including 4×10⁸ of AAV copies (Genome Copies (GC)) as a DNase digestion control) for plotting a standard curve were added to each tube, corresponding to a 10-fold dilution. The PCR tubes were incubated at 37 °C for 30 min. After digestion, 5 μL of a digested sample was taken and added to 95 μL of water for dilution, and this dilution was then repeated once more. In this way, the sample was diluted 80,000-fold in total. RefAAV (plasmid standard) was serially diluted 4,000-fold.

(2) SYBR Green qPCR

Standard preparation: A plasmid standard including 2×10⁸ AAV copies/L was subjected to 6 serial dilutions with 8 μL of the standard+72 μL of water for each dilution. The first concentration was 2×10⁸ GC/L, which was set to 8×10¹⁴ GC/mL in software to reflect the dilution series of the sample. Subsequent concentrations were 8×10¹³ GC/mL, 8×10¹² GC/mL, 8×10¹¹ GC/mL, 8×10¹⁰ GC/mL, and 8×10⁹ GC/mL, respectively.

Components of a reaction system were shown in Table 6:

TABLE 6
Reaction system

| Reagent | Vol. per reaction | |
|---|---|---|
| SYBR PCR reagent (2×) | 10 | μL |
| ROX(50×) | 0.4 | μL |
| FWD ITR(50 μM) | 0.1 | μL |
| REV ITR(50 μM) | 0.1 | μL |
| Nuclease-free water | 4.4 | μL |
| Sample DNA | 5 | μL |

With 3 replicate wells per sample, a corresponding volume of a mixture was prepared. 18 L of the mixture was added to each well, and then 2 μL of a sample was added to each well.

(3) Conditions for SYBR Green qPCR
Pre-denaturation: 95 °C for 10 min; and
Cycling: 40 cycles: 95 °C for 15 s and 60 °C for 1 min.

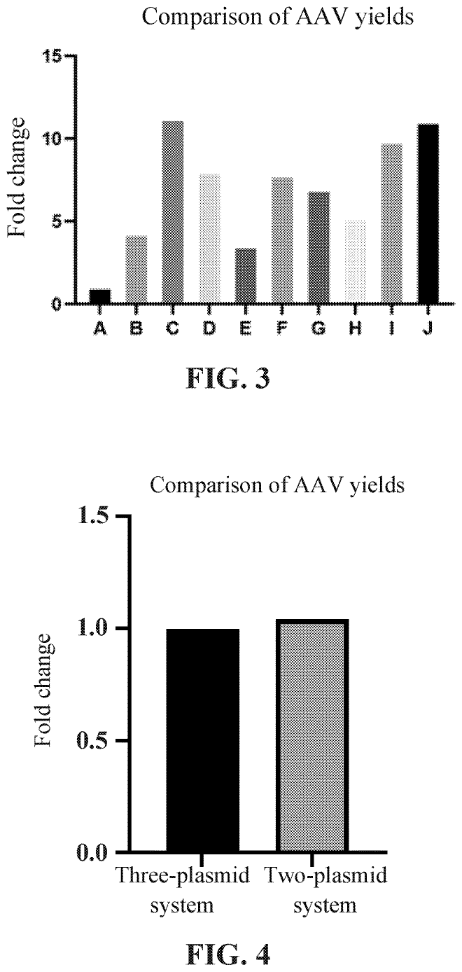

All titer test results were converted into fold changes relative to the control vector P5+RC (RepCap)+Helper (E2A/E4/VA RNA), as shown in FIG. 3. According to test results, compared with the pRCHelper helper vector “P5+RC+Helper” (group A) driven by a single P5 promoter, the helper vector “RC+Helper+P5” (group B) in which a P5 promoter was located downstream of RC+Helper increased the AAV yield by approximately 4.2-fold. The helper vectors “RC+Helper+DA′+P5” (group C) and “RC+DA′+Helper+P5” (group H) in which a DA′ sequence was additionally added could increase the AAV yield by approximately 11.2-fold and 5.2-fold, respectively. The helper vector “Helper+RC+DA′+P5” (group D) in which positions of RC and Helper were changed could increase the AAV yield by approximately 8-fold. The helper vector “RC+P5+Helper” (group E) in which a P5 promoter was located between RC and Helper could increase the AAV yield by approximately 3.5-fold. The helper vectors “RC+DA′+P5+Helper” (group F) and “RC+P5+Helper+DA′” (group G) in which a DA′ sequence was additionally added could increase the AAV yield by approximately 7.8-fold and 6.9-fold, respectively. The helper vectors “RC+DA′+Helper+DA′+P5” (group I) and “RC+DA′+P5+Helper+DA′+P5” (group J) in which two DA′ sequences were additionally added could increase the AAV yield by approximately 9.8-fold and 11-fold, respectively. The above results indicate that the helper plasmids constructed in Example 1 of the present application all can enhance the rAAV yield.

The optimal helper vector “RC+Helper+DA′+P5” constructed above and the packaging vector “RC+DA′+P5” from a preferred embodiment in U.S. Ser. No. 12/466,858B2 were each used in two-plasmid and three-plasmid systems to produce AAV. Titer results were converted into fold changes relative to the three-plasmid group as a control, as shown in FIG. 4. Results showed that the helper vector “RC+Helper+DA′+P5” in the two-plasmid group led to a higher rAAV yield than the packaging vector “RC+DA′+P5” in the three-plasmid group. That is, the titer of AAV produced by the two-plasmid system of the present application could reach or even exceed the titer of AAV produced by the three-plasmid system. In the examples, a helper plasmid of a specific serotype (Rep2Cap9) (a chimeric Rep/Cap plasmid in which a Rep gene is derived from AAV2, a Cap gene is derived from AAV9, and there is no spacer between the Rep gene and the Cap gene (directly adjacent)) is employed. It should be understood by those skilled in the art that the present application is not limited to this specific serotype, but can be practiced using other serotype helper plasmids that are currently known or may be discovered in the future.

In the present application, a nucleotide sequence (SEQ ID NO: 59) of the plasmid including the RepCap gene is as follows:

CCGCCATGCCGGGGTTTTACGAGATTGTGATTAAGGTCCCCAGCGACCTT
GACGAGCATCTGCCCGGCATTTCTGACAGCTTTGTGAACTGGGTGGCCGA
GAAGGAATGGGAGTTGCCGCCAGATTCTGACATGGATCTGAATCTGATTG
AGCAGGCACCCCTGACCGTGGCCGAGAAGCTGCAGCGCGACTTTCTGACG
GAATGGCGCCGTGTGAGTAAGGCCCCGGAGGCCCTTTTCTTTGTGCAATT
TGAGAAGGGAGAGAGCTACTTCCACATGCACGTGCTCGTGGAAACCACCG
GGGTGAAATCCATGGTTTTGGGACGTTTCCTGAGTCAGATTCGCGAAAAA
CTGATTCAGAGAATTTACCGCGGGATCGAGCCGACTTTGCCAAACTGGTT
CGCGGTCACAAAGACCAGAAATGGCGCCGGAGGCGGGAACAAGGTGGTGG
ATGAGTGCTACATCCCCAATTACTTGCTCCCCAAAACCCAGCCTGAGCTC
CAGTGGGCGTGGACTAATATGGAACAGTATTTAAGCGCCTGTTTGAATCT
CACGGAGCGTAAACGGTTGGTGGCGCAGCATCTGACGCACGTGTCGCAGA
CGCAGGAGCAGAACAAAGAGAATCAGAATCCCAATTCTGATGCGCCGGTG
ATCAGATCAAAAACTTCAGCCAGGTACATGGAGCTGGTCGGGTGGCTCGT
GGACAAGGGGATTACCTCGGAGAAGCAGTGGATCCAGGAGGACCAGGCCT
CATACATCTCCTTCAATGCGGCCTCCAACTCGCGGTCCCAAATCAAGGCT
GCCTTGGACAATGCGGGAAAGATTATGAGCCTGACTAAAACCGCCCCCGA
CTACCTGGTGGGCCAGCAGCCCGTGGAGGACATTTCCAGCAATCGGATTT
ATAAAATTTTGGAACTAAACGGGTACGATCCCCAATATGCGGCTTCCGTC
TTTCTGGGATGGGCCACGAAAAAGTTCGGCAAGAGGAACACCATCTGGCT
GTTTGGGCCTGCAACTACCGGGAAGACCAACATCGCGGAGGCCATAGCCC
ACACTGTGCCCTTCTACGGGTGCGTAAACTGGACCAATGAGAACTTTCCC
TTCAACGACTGTGTCGACAAGATGGTGATCTGGTGGGAGGAGGGGAAGAT
GACCGCCAAGGTCGTGGAGTCGGCCAAAGCCATTCTCGGAGGAAGCAAGG
TGCGCGTGGACCAGAAATGCAAGTCCTCGGCCCAGATAGACCCGACTCCC
GTGATCGTCACCTCCAACACCAACATGTGCGCCGTGATTGACGGGAACTC
AACGACCTTCGAACACCAGCAGCCGTTGCAAGACCGGATGTTCAAATTTG
AACTCACCCGCCGTCTGGATCATGACTTTGGGAAGGTCACCAAGCAGGAA
GTCAAAGACTTTTTCCGGTGGGCAAAGGATCACGTGGTTGAGGTGGAGCA
TGAATTCTACGTCAAAAAGGGTGGAGCCAAGAAAAGACCCGCCCCCAGTG
ACGCAGATATAAGTGAGCCCAAACGGGTGCGCGAGTCAGTTGCGCAGCCA
TCGACGTCAGACGCGGAAGCTTCGATCAACTACGCAGACAGGTACCAAAA
CAAATGTTCTCGTCACGTGGGCATGAATCTGATGCTGTTTCCCTGCAGAC
AATGCGAGAGAATGAATCAGAATTCAAATATCTGCTTCACTCACGGACAG
AAAGACTGTTTAGAGTGCTTTCCCGTGTCAGAATCTCAACCCGTTTCTGT
CGTCAAAAAGGCGTATCAGAAACTGTGCTACATTCATCATATCATGGGAA
AGGTGCCAGACGCTTGCACTGCCTGCGATCTGGTCAATGTGGATTTGGAT
GACTGCATCTTTGAACAATAAATGATTTAAATCAGGTATGGCTGCCGATG
GTTATCTTCCAGATTGGCTCGAGGACAACCTTAGTGAAGGAATTCGCGAG
TGGTGGGCTTTGAAACCTGGAGCCCCTCAACCCAAGGCAAATCAACAACA
TCAAGACAACGCTCGAGGTCTTGTGCTTCCGGGTTACAAATACCTTGGAC
CCGGCAACGGACTCGACAAGGGGGAGCCGGTCAACGCAGCAGACGCGGCG
GCCCTCGAGCACGACAAGGCCTACGACCAGCAGCTCAAGGCCGGAGACAA
CCCGTACCTCAAGTACAACCACGCCGACGCCGAGTTCCAGGAGCGGCTCA
AAGAAGATACGTCTTTTGGGGGCAACCTCGGGCGAGCAGTCTTCCAGGCC
AAAAAGAGGCTTCTTGAACCTCTTGGTCTGGTTGAGGAAGCGGCTAAGAC
GGCTCCTGGAAAGAAGAGGCCTGTAGAGCAGTCTCCTCAGGAACCGGACT
CCTCCGCGGGTATTGGCAAATCGGGTGCACAGCCCGCTAAAAAGAGACTC
AATTTCGGTCAGACTGGCGACACAGAGTCAGTCCCAGACCCTCAACCAAT
CGGAGAACCTCCCGCAGCCCCCTCAGGTGTGGGATCTCTTACAATGGCTT
CAGGTGGTGGCGCACCAGTGGCAGACAATAACGAAGGTGCCGATGGAGTG
GGTAGTTCCTCGGGAAATTGGCATTGCGATTCCCAATGGCTGGGGGACAG
AGTCATCACCACCAGCACCCGAACCTGGGCCCTGCCCACCTACAACAATC
ACCTCTACAAGCAAATCTCCAACAGCACATCTGGAGGATCTTCAAATGAC
AACGCCTACTTCGGCTACAGCACCCCCTGGGGGTATTTTGACTTCAACAG
ATTCCACTGCCACTTCTCACCACGTGACTGGCAGCGACTCATCAACAACA
ACTGGGGATTCCGGCCTAAGCGACTCAACTTCAAGCTCTTCAACATTCAG
GTCAAAGAGGTTACGGACAACAATGGAGTCAAGACCATCGCCAATAACCT
TACCAGCACGGTCCAGGTCTTCACGGACTCAGACTATCAGCTCCCGTACG
TGCTCGGGTCGGCTCACGAGGGCTGCCTCCCGCCGTTCCCAGCGGACGTT
TTCATGATTCCTCAGTACGGGTATCTGACGCTTAATGATGGAAGCCAGGC
CGTGGGTCGTTCGTCCTTTTACTGCCTGGAATATTTCCCGTCGCAAATGC
TAAGAACGGGTAACAACTTCCAGTTCAGCTACGAGTTTGAGAACGTACCT
TTCCATAGCAGCTACGCTCACAGCCAAAGCCTGGACCGACTAATGAATCC
ACTCATCGACCAATACTTGTACTATCTCTCAAAGACTATTAACGGTTCTG
GACAGAATCAACAAACGCTAAAATTCAGTGTGGCCGGACCCAGCAACATG
GCTGTCCAGGGAAGAAACTACATACCTGGACCCAGCTACCGACAACAACG
TGTCTCAACCACTGTGACTCAAAACAACAACAGCGAATTTGCTTGGCCTG
GAGCTTCTTCTTGGGCTCTCAATGGACGTAATAGCTTGATGAATCCTGGA
CCTGCTATGGCCAGCCACAAAGAAGGAGAGGACCGTTTCTTTCCTTTGTC
TGGATCTTTAATTTTTGGCAAACAAGGAACTGGAAGAGACAACGTGGATG
CGGACAAAGTCATGATAACCAACGAAGAAGAAATTAAAACTACTAACCCG
GTAGCAACGGAGTCCTATGGACAAGTGGCCACAAACCACCAGAGTGCCCA
AGCACAGGCGCAGACCGGCTGGGTTCAAAACCAAGGAATACTTCCGGGTA
TGGTTTGGCAGGACAGAGATGTGTACCTGCAAGGACCCATTTGGGCCAAA
ATTCCTCACACGGACGGCAACTTTCACCCTTCTCCGCTGATGGGAGGGTT
TGGAATGAAGCACCCGCCTCCTCAGATCCTCATCAAAAACACACCTGTAC
CTGCGGATCCTCCAACGGCCTTCAACAAGGACAAGCTGAACTCTTTCATC
ACCCAGTATTCTACTGGCCAAGTCAGCGTGGAGATCGAGTGGGAGCTGCA
GAAGGAAAACAGCAAGCGCTGGAACCCGGAGATCCAGTACACTTCCAACT
ATTACAAGTCTAATAATGTTGAATTTGCTGTTAATACTGAAGGTGTATAT
AGTGAACCCCGCCCCATTGGCACCAGATACCTGACTCGTAATCTGTAATT
GCCTGTTAATCAATAAACCGGTTGATTCGTTTCAGTTGAACTTTGGTCTC
TGCGAAGGGCGAATTCGTTTAAACCTGCAAGGAACCCCTAGTGATGGAGT
TGGCCACTCCCTCTCTGCGCGCTCGCTCGCTCACTGAGGCTGCAGGACTA
GAGGTCCTGTATTAGAGGTCACGTGAGTGTTTTGCGACATTTTGCGACAC
CATGTGGTCACGCTGGGTATTTAAGCCCGAGTGAGCACGCAGGGTCTCCA
TTTTGAAGCGGGAGGTTTGAACGCGCAGCCGCCAAGCCGAATTCTGCAGA
TATCCATCACACTGGCGGCCGCTCGACTAGAGCGGCCGCCACCGCGGTGG
AGCTCCAGCTTTTGTTCCCTTTAGTGAGGGTTAATTGCGCGCTTGGCGTA
ATCATGGTCATAGCTGTTTCCTGTGTGAAATTGTTATCCGCTCACAATTC
CACACAACATACGAGCCGGAAGCATAAAGTGTAAAGCCTGGGGTGCCTAA
TGAGTGAGCTAACTCACATTAATTGCGTTGCGCTCACTGCCCGCTTTCCA
GTCGGGAAACCTGTCGTGCCAGCTGCATTAATGAATCGGCCAACGCGCGG
GGAGAGGCGGTTTGCGTATTGGGCGCTCTTCCGCTTCCTCGCTCACTGAC
TCGCTGCGCTCGGTCGTTCGGCTGCGGCGAGCGGTATCAGCTCACTCAAA
GGCGGTAATACGGTTATCCACAGAATCAGGGGATAACGCAGGAAAGAACA
TGTGAGCAAAAGGCCAGCAAAAGGCCAGGAACCGTAAAAAGGCCGCGTTG
CTGGCGTTTTTCCATAGGCTCCGCCCCCCTGACGAGCATCACAAAAATCG
ACGCTCAAGTCAGAGGTGGCGAAACCCGACAGGACTATAAAGATACCAGG
CGTTTCCCCCTGGAAGCTCCCTCGTGCGCTCTCCTGTTCCGACCCTGCCG
CTTACCGGATACCTGTCCGCCTTTCTCCCTTCGGGAAGCGTGGCGCTTTC
TCATAGCTCACGCTGTAGGTATCTCAGTTCGGTGTAGGTCGTTCGCTCCA
AGCTGGGCTGTGTGCACGAACCCCCCGTTCAGCCCGACCGCTGCGCCTTA
TCCGGTAACTATCGTCTTGAGTCCAACCCGGTAAGACACGACTTATCGCC
ACTGGCAGCAGCCACTGGTAACAGGATTAGCAGAGCGAGGTATGTAGGCG
GTGCTACAGAGTTCTTGAAGTGGTGGCCTAACTACGGCTACACTAGAAGA
ACAGTATTTGGTATCTGCGCTCTGCTGAAGCCAGTTACCTTCGGAAAAAG
AGTTGGTAGCTCTTGATCCGGCAAACAAACCACCGCTGGTAGCGGTGGTT
TTTTTGTTTGCAAGCAGCAGATTACGCGCAGAAAAAAAGGATCTCAAGAA
GATCCTTTGATCTTTTCTACGGGGTCTGACGCTCAGTGGAACGAAAACTC
ACGTTAAGGGATTTTGGTCATGAGATTATCAAAAAGGATCTTCACCTAGA
TCCTTTTAAATTAAAAATGAAGTTTTAAATCAATCTAAAGTATATATGAG
TAAACTTGGTCTGACAGTTACCAATGCTTAATCAGTGAGGCACCTATCTC
AGCGATCTGTCTATTTCGTTCATCCATAGTTGCCTGACTCCCCGTCGTGT
AGATAACTACGATACGGGAGGGCTTACCATCTGGCCCCAGTGCTGCAATG
ATACCGCGAGACCCACGCTCACCGGCTCCAGATTTATCAGCAATAAACCA
GCCAGCCGGAAGGGCCGAGCGCAGAAGTGGTCCTGCAACTTTATCCGCCT
CCATCCAGTCTATTAATTGTTGCCGGGAAGCTAGAGTAAGTAGTTCGCCA
GTTAATAGTTTGCGCAACGTTGTTGCCATTGCTACAGGCATCGTGGTGTC
ACGCTCGTCGTTTGGTATGGCTTCATTCAGCTCCGGTTCCCAACGATCAA
GGCGAGTTACATGATCCCCCATGTTGTGCAAAAAAGCGGTTAGCTCCTTC
GGTCCTCCGATCGTTGTCAGAAGTAAGTTGGCCGCAGTGTTATCACTCAT
GGTTATGGCAGCACTGCATAATTCTCTTACTGTCATGCCATCCGTAAGAT
GCTTTTCTGTGACTGGTGAGTACTCAACCAAGTCATTCTGAGAATAGTGT
ATGCGGCGACCGAGTTGCTCTTGCCCGGCGTCAATACGGGATAATACCGC
GCCACATAGCAGAACTTTAAAAGTGCTCATCATTGGAAAACGTTCTTCGG
GGCGAAAACTCTCAAGGATCTTACCGCTGTTGAGATCCAGTTCGATGTAA
CCCACTCGTGCACCCAACTGATCTTCAGCATCTTTTACTTTCACCAGCGT
TTCTGGGTGAGCAAAAACAGGAAGGCAAAATGCCGCAAAAAAGGGAATAA
GGGCGACACGGAAATGTTGAATACTCATACTCTTCCTTTTTCAATATTAT
TGAAGCATTTATCAGGGTTATTGTCTCATGAGCGGATACATATTTGAATG
TATTTAGAAAAATAAACAAATAGGGGTTCCGCGCACATTTCCCCGAAAAG
TGCCACCTAAATTGTAAGCGTTAATATTTTGTTAAAATTCGCGTTAAATT
TTTGTTAAATCAGCTCATTTTTTAACCAATAGGCCGAAATCGGCAAAATC
CCTTATAAATCAAAAGAATAGACCGAGATAGGGTTGAGTGTTGTTCCAGT
TTGGAACAAGAGTCCACTATTAAAGAACGTGGACTCCAACGTCAAAGGGC
GAAAAACCGTCTATCAGGGCGATGGCCCACTACGTGAACCATCACCCTAA
TCAAGTTTTTTGGGGTCGAGGTGCCGTAAAGCACTAAATCGGAACCCTAA
AGGGAGCCCCCGATTTAGAGCTTGACGGGGAAAGCCGGCGAACGTGGCGA
GAAAGGAAGGGAAGAAAGCGAAAGGAGCGGGCGCTAGGGCGCTGGCAAGT
GTAGCGGTCACGCTGCGCGTAACCACCACACCCGCCGCGCTTAATGCGCC
GCTACAGGGCGCGTCCCATTCGCCATTCAGGCTGCGCAACTGTTGGGAAG
GGCGATCGGTGCGGGCCTCTTCGCTATTACGCCAGCTGGCGAAAGGGGGA
TGTGCTGCAAGGCGATTAAGTTGGGTAACGCCAGGGTTTTCCCAGTCACG
ACGTTGTAAAACGACGGCCAGTGAGCGCGCGTAATACGACTCACTATAGG
GCGAATTGGGTACCGGGCCCCCCCTCGATCGAGGTCGACGGTATCGGGGG
AGCTCGCAGGGTCTCCATTTTGAAGCGGGAGGTTTGAACGCGCAG.

In the present application, a nucleotide sequence (SEQ ID NO: 60) of the pAAV.CAG.EGFP vector is as follows:

CCTGCAGGCAGCTGCGCGCTCGCTCGCTCACTGAGGCCGCCCGGGCGTCG
GGCGACCTTTGGTCGCCCGGCCTCAGTGAGCGAGCGAGCGCGCAGAGAGG
GAGTGGCCAACTCCATCACTAGGGGTTCCTGCGGCCATTCGGTACAATTC
ACGCGTCGACATTGATTATTGACTAGCTCTGGTCGTTACATAACTTACGG
TAAATGGCCCGCCTGGCTGACCGCCCAACGACCCCCGCCCATTGACGTCA
ATAATGACGTATGTTCCCATAGTAACGCCAATAGGGACTTTCCATTGACG
TCAATGGGTGGAGTATTTACGGTAAACTGCCCACTTGGCAGTACATCAAG
TGTATCATATGCCAAGTACGCCCCCTATTGACGTCAATGACGGTAAATGG
CCCGCCTGGCATTATGCCCAGTACATGACCTTATGGGACTTTCCTACTTG
GCAGTACATCTACTCGAGGCCACGTTCTGCTTCACTCTCCCCATCTCCCC
CCCCTCCCCACCCCCAATTTTGTATTTATTTATTTTTTAATTATTTTGTG
CAGCGATGGGGGCGGGGGGGGGGGGGGGGCGCGCGCCAGGCGGGGCGGGG
CGGGGCGAGGGGGGGGCGGGGCGAGGCGGAGAGGTGCGGCGGCAGCCAAT
CAGAGCGGCGCGCTCCGAAAGTTTCCTTTTATGGCGAGGCGGCGGCGGCG
GCGGCCCTATAAAAAGCGAAGCGCGCGGCGGGCGGGAGCGGGATCAGCCA
CCGCGGTGGCGGCCTAGAGTCGACGAGGAACTGAAAAACCAGAAAGTTAA
CTGGTAAGTTTAGTCTTTTTGTCTTTTATTTCAGGTCCCGGATCCGGTGG
TGGTGCAAATCAAAGAACTGCTCCTCAGTGGATGTTGCCTTTACTTCTAG
GCCTGTACGGAAGTGTTACTTCTGCTCTAAAAGCTGCGGAATTGTACCCG
CGGCCGATCCACCGGTCGCCACCATGGTGAGCAAGGGCGAGGAGCTGTTC
ACCGGGGTGGTGCCCATCCTGGTCGAGCTGGACGGCGACGTAAACGGCCA
CAAGTTCAGCGTGTCCGGCGAGGGCGAGGGCGATGCCACCTACGGCAAGC
TGACCCTGAAGTTCATCTGCACCACCGGCAAGCTGCCCGTGCCCTGGCCC
ACCCTCGTGACCACCCTGACCTACGGCGTGCAGTGCTTCAGCCGCTACCC
CGACCACATGAAGCAGCACGACTTCTTCAAGTCCGCCATGCCCGAAGGCT
ACGTCCAGGAGCGCACCATCTTCTTCAAGGACGACGGCAACTACAAGACC
CGCGCCGAGGTGAAGTTCGAGGGCGACACCCTGGTGAACCGCATCGAGCT
GAAGGGCATCGACTTCAAGGAGGACGGCAACATCCTGGGGCACAAGCTGG
AGTACAACTACAACAGCCACAACGTCTATATCATGGCCGACAAGCAGAAG
AACGGCATCAAGGTGAACTTCAAGATCCGCCACAACATCGAGGACGGCAG
CGTGCAGCTCGCCGACCACTACCAGCAGAACACCCCCATCGGCGACGGCC
CCGTGCTGCTGCCCGACAACCACTACCTGAGCACCCAGTCCGCCCTGAGC
AAAGACCCCAACGAGAAGCGCGATCACATGGTCCTGCTGGAGTTCGTGAC
CGCCGCCGGGATCACTCTCGGCATGGACGAGCTGTACAAGTAAATCGAAT
TCCGCTCGAGATAATCAACCTCTGGATTACAAAATTTGTGAAAGATTGAC
TGGTATTCTTAACTATGTTGCTCCTTTTACGCTATGTGGATACGCTGCTT
TAATGCCTTTGTATCATGCTATTGCTTCCCGTATGGCTTTCATTTTCTCC
TCCTTGTATAAATCCTGGTTAGTTCTTGCCACGGCGGAACTCATCGCCGC
CTGCCTTGCCCGCTGCTGGACAGGGGCTCGGCTGTTGGGCACTGACAATT
CCGTGGTGTTTATTTGTGAAATTTGTGATGCTATTGCTTTATTTGTAACC
ATCTAGCTTTATTTGTGAAATTTGTGATGCTATTGCTTTATTTGTAACCA
TTATAAGCTGCAATAAACAAGTTAACAACAACAATTGCATTCATTTTATG
TTTCAGGTTCAGGGGGAGATGTGGGAGGTTTTTTAAAGCGGCCGCAGGAA
CCCCTAGTGATGGAGTTGGCCACTCCCTCTCTGCGCGCTCGCTCGCTCAC
TGAGGCCGGGCGACCAAAGGTCGCCCGACGCCCGGGCTTTGCCCGGGCGG
CCTCAGTGAGCGAGCGAGCGCGCAGCTGCCTGCAGGGGCGCCTGATGCGG
TATTTTCTCCTTACGCATCTGTGCGGTATTTCACACCGCATACGTCAAAG
CAACCATAGTACGCGCCCTGTAGCGGCGCATTAAGCGCGGCGGGTGTGGT
GGTTACGCGCAGCGTGACCGCTACACTTGCCAGCGCCCTAGCGCCCGCTC
CTTTCGCTTTCTTCCCTTCCTTTCTCGCCACGTTCGCCGGCTTTCCCCGT
CAAGCTCTAAATCGGGGGCTCCCTTTAGGGTTCCGATTTAGTGCTTTACG
GCACCTCGACCCCAAAAAACTTGATTTGGGTGATGGTTCACGTAGTGGGC
CATCGCCCTGATAGACGGTTTTTCGCCCTTTGACGTTGGAGTCCACGTTC
TTTAATAGTGGACTCTTGTTCCAAACTGGAACAACACTCAACCCTATCTC
GGGCTATTCTTTTGATTTATAAGGGATTTTGCCGATTTCGGCCTATTGGT
TAAAAAATGAGCTGATTTAACAAAAATTTAACGCGAATTTTAACAAAATA
TTAACGTTTACAATTTTATGGTGCACTCTCAGTACAATCTGCTCTGATGC
CGCATAGTTAAGCCAGCCCCGACACCCGCCAACACCCGCTGACGCGCCCT
GACGGGCTTGTCTGCTCCCGGCATCCGCTTACAGACAAGCTGTGACCGTC
TCCGGGAGCTGCATGTGTCAGAGGTTTTCACCGTCATCACCGAAACGCGC
GAGACGAAAGGGCCTCGTGATACGCCTATTTTTATAGGTTAATGTCATGA
TAATAATGGTTTCTTAGACGTCAGGTGGCACTTTTCGGGGAAATGTGCGC
GGAACCCCTATTTGTTTATTTTTCTAAATACATTCAAATATGTATCCGCT
CATGAGACAATAACCCTGATAAATGCTTCAATAATATTGAAAAAGGAAGA
GTATGAGTATTCAACATTTCCGTGTCGCCCTTATTCCCTTTTTTGCGGCA
TTTTGCCTTCCTGTTTTTGCTCACCCAGAAACGCTGGTGAAAGTAAAAGA
TGCTGAAGATCAGTTGGGTGCACGAGTGGGTTACATCGAACTGGATCTCA
ACAGCGGTAAGATCCTTGAGAGTTTTCGCCCCGAAGAACGTTTTCCAATG
ATGAGCACTTTTAAAGTTCTGCTATGTGGCGCGGTATTATCCCGTATTGA
CGCCGGGCAAGAGCAACTCGGTCGCCGCATACACTATTCTCAGAATGACT
TGGTTGAGTACTCACCAGTCACAGAAAAGCATCTTACGGATGGCATGACA
GTAAGAGAATTATGCAGTGCTGCCATAACCATGAGTGATAACACTGCGGC
CAACTTACTTCTGACAACGATCGGAGGACCGAAGGAGCTAACCGCTTTTT
TGCACAACATGGGGGATCATGTAACTCGCCTTGATCGTTGGGAACCGGAG
CTGAATGAAGCCATACCAAACGACGAGCGTGACACCACGATGCCTGTAGC
AATGGCAACAACGTTGCGCAAACTATTAACTGGCGAACTACTTACTCTAG
CTTCCCGGCAACAATTAATAGACTGGATGGAGGCGGATAAAGTTGCAGGA
CCACTTCTGCGCTCGGCCCTTCCGGCTGGCTGGTTTATTGCTGATAAATC
TGGAGCCGGTGAGCGTGGGTCTCGCGGTATCATTGCAGCACTGGGGCCAG
ATGGTAAGCCCTCCCGTATCGTAGTTATCTACACGACGGGGAGTCAGGCA
ACTATGGATGAACGAAATAGACAGATCGCTGAGATAGGTGCCTCACTGAT
TAAGCATTGGTAACTGTCAGACCAAGTTTACTCATATATACTTTAGATTG
ATTTAAAACTTCATTTTTAATTTAAAAGGATCTAGGTGAAGATCCTTTTT
GATAATCTCATGACCAAAATCCCTTAACGTGAGTTTTCGTTCCACTGAGC
GTCAGACCCCGTAGAAAAGATCAAAGGATCTTCTTGAGATCCTTTTTTTC
TGCGCGTAATCTGCTGCTTGCAAACAAAAAAACCACCGCTACCAGCGGTG
GTTTGTTTGCCGGATCAAGAGCTACCAACTCTTTTTCCGAAGGTAACTGG
CTTCAGCAGAGCGCAGATACCAAATACTGTCCTTCTAGTGTAGCCGTAGT
TAGGCCACCACTTCAAGAACTCTGTAGCACCGCCTACATACCTCGCTCTG
CTAATCCTGTTACCAGTGGCTGCTGCCAGTGGCGATAAGTCGTGTCTTAC
CGGGTTGGACTCAAGACGATAGTTACCGGATAAGGCGCAGCGGTCGGGCT
GAACGGGGGGTTCGTGCACACAGCCCAGCTTGGAGCGAACGACCTACACC
GAACTGAGATACCTACAGCGTGAGCTATGAGAAAGCGCCACGCTTCCCGA
AGGGAGAAAGGCGGACAGGTATCCGGTAAGCGGCAGGGTCGGAACAGGAG
AGCGCACGAGGGAGCTTCCAGGGGGAAACGCCTGGTATCTTTATAGTCCT
GTCGGGTTTCGCCACCTCTGACTTGAGCGTCGATTTTTGTGATGCTCGTC
AGGGGGGCGGAGCCTATGGAAAAACGCCAGCAACGCGGCCTTTTTACGGT
TCCTGGCCTTTTGCTGGCCTTTTGCTCACATGT.

Finally, it should be noted that the above examples are provided merely to describe the technical solutions of the present application, rather than to limit the scope of protection of the present application. Although the present application is described in detail with reference to preferred examples, those of ordinary skill in the art should understand that modifications or equivalent replacements may be made to the technical solutions of the present application without departing from the spirit and scope of the technical solutions of the present application.

1. A helper plasmid for producing a recombinant adeno-associated virus (rAAV), comprising:
a backbone plasmid and a recombination sequence,
wherein the recombination sequence comprises:
a Rep protein-encoding sequence, a Cap protein-encoding sequence, an E2A sequence, an E4 sequence, a VA RNA-encoding DNA sequence, and at least one promoter sequence; and
the at least one promoter sequence is located downstream of the Rep protein-encoding sequence and the Cap protein-encoding sequence.

2. The helper plasmid according to claim 1, wherein the recombination sequence further comprises at least one DA′ sequence.

3. The helper plasmid according to claim 2, wherein the at least one DA′ sequence is located downstream of the Rep protein-encoding sequence and the Cap protein-encoding sequence.

4. The helper plasmid according to claim 2, wherein the recombination sequence comprises, in a 5′ to 3′ direction, any one selected from the group consisting of following combinations:
i. the Rep protein-encoding sequence, the Cap protein-encoding sequence, the E2A sequence, the E4 sequence, the VA RNA-encoding DNA sequence, and a first promoter sequence;
ii. the Rep protein-encoding sequence, the Cap protein-encoding sequence, the E2A sequence, the E4 sequence, the VA RNA-encoding DNA sequence, the DA′ sequence, and the first promoter sequence;
iii. the E2A sequence, the E4 sequence, the VA RNA-encoding DNA sequence, the Rep protein-encoding sequence, the Cap protein-encoding sequence, the DA′ sequence, and the first promoter sequence;
iv. the Rep protein-encoding sequence, the Cap protein-encoding sequence, the first promoter sequence, the E2A sequence, the E4 sequence, and the VA RNA-encoding DNA sequence;
v. the Rep protein-encoding sequence, the Cap protein-encoding sequence, the DA′ sequence, the first promoter sequence, the E2A sequence, the E4 sequence, and the VA RNA-encoding DNA sequence;
vi. the Rep protein-encoding sequence, the Cap protein-encoding sequence, the first promoter sequence, the E2A sequence, the E4 sequence, the VA RNA-encoding DNA sequence, and the DA′ sequence;
vii. the Rep protein-encoding sequence, the Cap protein-encoding sequence, the DA′ sequence, the E2A sequence, the E4 sequence, the VA RNA-encoding DNA sequence, and the first promoter sequence;
viii. the Rep protein-encoding sequence, the Cap protein-encoding sequence, the DA′ sequence, the E2A sequence, the E4 sequence, the VA RNA-encoding DNA sequence, the DA′ sequence, and the first promoter sequence; and
ix. the Rep protein-encoding sequence, the Cap protein-encoding sequence, the DA′ sequence, the first promoter sequence, the E2A sequence, the E4 sequence, the VA RNA-encoding DNA sequence, the DA′ sequence, and a second promoter sequence.

5. The helper plasmid according to claim 1, wherein the at least one promoter sequence comprises one or more P5 promoter sequences, and optionally, a P5 promoter sequence is set forth in SEQ ID NO: 2.

6. The helper plasmid according to claim 4, wherein the first promoter sequence and/or the second promoter sequence are/is a P5 promoter sequence, and optionally, the P5 promoter sequence is set forth in SEQ ID NO: 2.

7. The helper plasmid according to claim 1, wherein the Rep protein-encoding sequence is derived from adeno-associated virus (AAV) serotype 2 and the Cap protein-encoding sequence is derived from AAV serotype 9.

8. The helper plasmid according to claim 7, wherein the Rep protein-encoding sequence is set forth in SEQ ID NO: 4 and/or the Cap protein-encoding sequence is set forth in SEQ ID NO: 5.

9. The helper plasmid according to claim 1, wherein the E2A sequence, the E4 sequence, and the VA RNA-encoding DNA sequence are derived from an adenovirus.

10. The helper plasmid according to claim 9, wherein a nucleotide sequence of the E2A sequence is set forth in SEQ ID NO: 6, a nucleotide sequence of the E4 sequence is set forth in SEQ ID NO: 7, and the VA RNA-encoding DNA sequence is set forth in SEQ ID NO: 8.

11. The helper plasmid according to claim 4, wherein the first promoter sequence is set forth in SEQ ID NO: 2, the second promoter sequence is set forth in SEQ ID NO: 2, the DA′ sequence is set forth in SEQ ID NO: 3, the Rep protein-encoding sequence is set forth in SEQ ID NO: 4, the Cap protein-encoding sequence is set forth in SEQ ID NO: 5, a nucleotide sequence of the E2A sequence is set forth in SEQ ID NO: 6, a nucleotide sequence of the E4 sequence is set forth in SEQ ID NO: 7, and the VA RNA-encoding DNA sequence is set forth in SEQ ID NO: 8.

12. A plasmid combination for producing rAAV, comprising the helper plasmid according to claim 1 and an AAV plasmid.

13. A plasmid combination for producing rAAV, comprising the helper plasmid according to claim 4 and an AAV plasmid.

14. A plasmid combination for producing rAAV, comprising the helper plasmid according to claim 10 and an AAV plasmid.

15. A system for producing rAAV, wherein the system is prepared by transforming a host cell with the plasmid combination according to claim 12.

16. A system for producing rAAV, wherein the system is prepared by transforming a host cell with the plasmid combination according to claim 13.

17. A system for producing rAAV, wherein the system is prepared by transforming a host cell with the plasmid combination according to claim 14.

18. A method for producing rAAV, comprising: producing the rAAV using the system according to claim 15.

19. A method for producing rAAV, comprising: producing the rAAV using the system according to claim 16.

20. A method for producing rAAV, comprising: producing the rAAV using the system according to claim 17.


SEQUENCE LISTING (60 sequences)

SEQ ID NO: 1 - DNA, 2545 bp, synthetic construct
  source 1..2545: mol_type = other DNA
tttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggtttt

SEQ ID NO: 2 - DNA, 132 bp, Adeno-associated dependoparvovirus A
  source 1..132: mol_type = genomic DNA
aggtcctgtattagaggtcacgtgagtgttttgcgacattttgcgacaccatgtggtcacgctgggtatttaagcccgagtgagcacgcagggtctccattttgaagcgggaggtttgaacgcgcagccgcc

SEQ ID NO: 3 - DNA, 61 bp, Adeno-associated dependoparvovirus A
  source 1..61: mol_type = genomic DNA
aggaacccctagtgatggagttggccactccctctctgcgcgctcgctcgctcactgaggc

SEQ ID NO: 4 - DNA, 1563 bp, Adeno-associated dependoparvovirus A
  source 1..1563: mol_type = genomic DNA
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaa

SEQ ID NO: 5 - DNA, 2211 bp, Adeno-associated dependoparvovirus A
  source 1..2211: mol_type = genomic DNA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 6 - DNA, 5336 bp, Human mastadenovirus C
  source 1..5336: mol_type = genomic DNA
cgaccgcaccctgtgacgaaagccgcccgcaagctgcgcccctgagttagtcatctgaacttcggcctgggcgtctctgggaagtaccacagtggtgggagcgggactttcctggtacaccagggcagcgggccaactacggggattaaggttattacgaggtgtggtggtaatagccgcctgttcgaggagaattcggtttcggtgggcgcggattccgttgacccgggatatcatgtggggtcccgcgctcatgtagtttattcgggttgagtagtcttgggcagctccagccgcaagtcccatttgtggctggtaactccacatgtagggcgtgggaatttccttgctcataatggcgctgacgacaggtgctggcgccgggtgtggccgctggagatgacgtagttttcgcgcttaaatttgagaaagggcgcgaaactagtccttaagagtcagcgcgcagtatttgctgaagagagcctccgcgtcttccagcgtgcgccgaagctgatcttcgcttttgtgatacaggcagctgcgggtgagggagcgcagagacctgttttttattttcagctcttgttcttggcccctgctttgttgaaatatagcatacagagtgggaaaaatcctatttctaagctcgcgggtcgatacgggttcgttgggcgccagacgcagcgctcctcctcctgctgctgccgccgctgtggatttcttgggctttgtcagagtcttgctatccggtcgcctttgcttctgtgtgaccgctgctgttgctgccgctgccgctgccgccggtgcagtaggggctgtagagatgacggtagtaatgcaggatgttacgggggaaggccacgccgtgatggtagagaagaaagcggcgggcgaaggagatgttgcccccacagtcttgcaagcaagcaactatggcgttcttgtgcccgcgccacgagcggtagccttggcgctgttgttgctcttgggctaacggcggcggctgcttagacttaccggccctggttccagtggtgtcccatctacggttgggtcggcgaacaggcagtgccggcggcgcctgaggagcggaggttgtagcgatgctgggaacggttgccaatttctggggcgccggcgaggggaatgcgaccgagggtgacggtgtttcgtctgacacctcttcggcctcggaagcttcgtctaggctgtcccagtcttccatcatctcctcctcctcgtccaaaacctcctctgcctgactgtcccagtattcctcctcgtccgtgggtggcggcggcggcagctgcagcttctttttgggtgccatcctgggaagcaagggcccgcggctgctgatagggctgcggcggcggggggattgggttgagctcctcgccggactgggggtccaggtaaaccccccgtccctttcgtagcagaaactcttggcgggctttgttgatggcttgcaattggccaaggatgtggccctgggtaatgacgcaggcggtaagctccgcatttggcgggcgggattggtcttcgtagaacctaatctcgtgggcgtggtagtcctcaggtacaaatttgcgaaggtaagccgacgtccacagccccggagtgagtttcaaccccggagccgcggacttttcgtcaggcgagggaccctgcagctcaaaggtaccgataatttgactttcgctaagcagttgcgaattgcagaccagggagcggtgcggggtgcataggttgcagcgacagtgacactccagtaggccgtcaccgctcacgtcttccatgatgtcggagtggtaggcaaggtagttggctagctgcagaaggtagcagtgaccccaaagcggcggagggcattcacggtacttaatgggcacaaagtcgctaggaagcgcacagcaggtggcgggcagaattcctgaacgctctaggataaagttcctaaagttttgcaacatgctttgactggtgaagtctggcagaccctgttgcagggttttaagcaggcgttcggggaagataatgtccgccaggtgcgcggccacggagcgctcgttgaaggccgtccataggtccttcaagttttgctttagcagcttctgcagctcctttaggttgcgctcctccaggcattgctgccacacgcccatggccgtttgccaggtgtagcacagaaataagtaaacgcagtcgcggacgtagtcgcggcgcgcctcgcccttgagcgtggaatgaagcacgttttgcccgaggcggttttcgtgcaaaattccaaggtaggagaccaggttgcagagctccacgttggaaattttgcaggcctggcgcacgtagccctggcgaaaggtgtagtgcaacgtttcctctagcttgcgctgcatctccgggtcagcaaagaaccgctgcatgcactcaagctccacggtaacaagcactgcggccatcattagcttgcgtcgctcctccaagtcggcaggctcgcgcgtctcaagccagcgcgccagctgctcatcgccaactgcgggtaggccctcctcggtttgttcttgcaagtttgcatccctctccaggggtcgtgcacggcgcacgatcagctcgctcatgactgtgctcataaccttggggggtaggttaagtgccgggtaggcaaagtgggtgacctcgatgctgcgtttcagcacggctaggcgcgcgttgtcaccctcaagttccaccagcactccacagtgactttcattttcgctgttttcttgttgcagagcgtttgccgcgcgtttctcgtcgcgtccaagaccctcaaagatttttggcacttcgtcgagcgaggcgatatcaggtatgacagcgccctgccgcaaggccagctgcttgtccgctcggctgcggttggcacggcaggataggggtatcttgcagttttggaaaaagatgtgataggtggcaagcacctctggcacggcaaatacggggtagaagttgaggcgcgggttgggctcgcatgtgccgttttcttggcgtttggggggtacgcgcggtgagaacaggtggcgttcgtaggcaaggctgacatccgctatggcgaggggcacatcgctgcgctcttgcaacgcgtcgcagataatggcgcactggcgctgcagatgcttcaacagcacgtcgtctcccacatctaggtagtcgccatgcctttggtccccccgcccgacttgttcctcgtttgcctctgcgtcgtcctggtcttgctttttatcctctgttggtactgagcgatcctcgtcgtcttcgcttacaaaacctgggtcctgctcgataatcacttcctcctcctcaagcgggggtgcctcgacggggaaggtggtaggcgcgttggcggcatcggtggaggcggtggtggcgaactcaaagggggcggttaggctgtcctccttctcgactgactccatgatctttttctgcctataggagaaggaaatggccagtcgggaagaggagcagcgcgaaaccacccccgagcgcggacgcggtgcggcgcgacgtccaccaaccatggaggacgtgtcgtccccgtcgccgtcgccgccgcctccccgcgcgcccccaaaaaagcggctgaggcggcgtctcgagtccgaggacgaagaagactcgtcacaagatgcgctggtgccgcgcacacccagcccgcggccatcgacctcgacggcggatttggccattgcgtccaaaaagaaaaagaagcgcccctctcccaagcccgagcgcccgccatccccagaggtgatcgtggacagcgaggaagaaagagaagatgtggcgctacaaatggtgggtttcagcaacccaccggtgctaatcaagcacggcaagggaggtaagcgcacggtgcggcggctgaatgaagacgacccagtggcgcggggtatgcggacgcaagaggaaaaggaagagtccagtgaagcggaaagtgaaagcacggtgataaacccgctgagcctgccgatcgtgtctgcgtgggagaagggcatggaggctgcgcgcgcgttgatggacaagtaccacgtggataacgatctaaaggcaaacttcaagctactgcctgaccaagtggaagctctggcggccgtatgcaagacctggctaaacgaggagcaccgcgggttgcagctgaccttcaccagcaacaagacctttgtgacgatgatggggcgattcctgcaggcgtacctgcagtcgtttgcagaggtaacctacaagcaccacgagcccacgggctgcgcgttgtggctgcaccgctgcgctgagatcgaaggcgagcttaagtgtctacacgggagcattatgataaataaggagcacgtgattgaaatggatgtgacgagcgaaaacgggcagcgcgcgctgaaggagcagtctagcaaggccaagatcgtgaagaaccggtggggccgaaatgtggtgcagatctccaacaccgacgcaaggtgctgcgtgcatgacgcggcctgtccggccaatcagttttccggcaagtcttgcggcatgttcttctctgaaggcgcaaaggctcaggtggcttttaagcagatcaaggctttcatgcaggcgctgtatcctaacgcccagaccgggcacggtcaccttctgatgccactacggtgcgagtgcaactcaaagcctgggcatgcaccctttttgggaaggcagctaccaaagttgactccgttcgccctgagcaacgcggaggacctggacgcggatctgatctccgacaagagcgtgctggccagcgtgcaccacccggcgctgatagtgttccagtgctgcaaccctgtgtatcgcaactcgcgcgcgcagggcggaggccccaactgcgacttcaagatatcggcgcccgacctgctaaacgcgttggtgatggtgcgcagcctgtggagtgaaaacttcaccgagctgccgcggatggttgtgcctgagtttaagtggagcactaaacaccagtatcgcaacgtgtccctgccagtggcgcatagcgatgcgcggcagaacccctttgatttttaaacggcgcagacggcaagggtggggggtaaataatcacccgagagtgtacaaataaaaacatttgcctttattgaaagtgtctcctagtacattatttttacatgtttttcaagtgacaaaaagaagtggcgctcctaatctgcgcactgtggctgcggaagtagggcgagtggcgctccaggaagctgtagagctgttcctggttgcgacgcagggtgggctgtacctggggactgttaagcatggagttgggtacc

SEQ ID NO: 7 - DNA, 3201 bp, Human mastadenovirus C
  source 1..3201: mol_type = genomic DNA
cccgggcgttttagggcggagtaacttgcatgtattgggaattgtagtttttttaaaatgggaagtgacgtatcgtgggaaaacggaagtgaagatttgaggaagttgtgggttttttggctttcgtttctgggcgtaggttcgcgtgcggttttctgggtgttttttgtggactttaaccgttacgtcattttttagtcctatatatactcgctctgtacttggccctttttacactgtgactgattgagctggtgccgtgtcgagtggtgttttttaataggtttttttactggtaaggctgactgttatggctgccgctgtggaagcgctgtatgttgttctggagcgggagggtgctattttgcctaggcaggagggtttttcaggtgtttatgtgtttttctctcctattaattttgttatacctcctatgggggctgtaatgttgtctctacgcctgcgggtatgtattcccccgggctatttcggtcgctttttagcactgaccgatgttaaccaacctgatgtgtttaccgagtcttacattatgactccggacatgaccgaggaactgtcggtggtgctttttaatcacggtgaccagtttttttacggtcacgccggcatggccgtagtccgtcttatgcttataagggttgtttttcctgttgtaagacaggcttctaatgtttaaatgtttttttttttgttattttattttgtgtttaatgcaggaacccgcagacatgtttgagagaaaaatggtgtctttttctgtggtggttccggaacttacctgcctttatctgcatgagcatgactacgatgtgcttgcttttttgcgcgaggctttgcctgattttttgagcagcaccttgcattttatatcgccgcccatgcaacaagcttacataggggctacgctggttagcatagctccgagtatgcgtgtcataatcagtgtgggttcttttgtcatggttcctggcggggaagtggccgcgctggtccgtgcagacctgcacgattatgttcagctggccctgcgaagggacctacgggatcgcggtatttttgttaatgttccgcttttgaatcttatacaggtctgtgaggaacctgaatttttgcaatcatgattcgctgcttgaggctgaaggtggagggcgctctggagcagatttttacaatggccggacttaatattcgggatttgcttagagacatattgataaggtggcgagatgaaaattatttgggcatggttgaaggtgctggaatgtttatagaggagattcaccctgaagggtttagcctttacgtccacttggacgtgagggcagtttgccttttggaagccattgtgcaacatcttacaaatgccattatctgttctttggctgtagagtttgaccacgccaccggaggggagcgcgttcacttaatagatcttcattttgaggttttggataatcttttggaataaaaaaaaaaaaacatggttcttccagctcttcccgctcctcccgtgtgtgactcgcagaacgaatgtgtaggttggctgggtgtggcttattctgcggtggtggatgttatcagggcagcggcgcatgaaggagtttacatagaacccgaagccagggggcgcctggatgctttgagagagtggatatactacaactactacacagagcgagctaagcgacgagaccggagacgcagatctgtttgtcacgcccgcacctggttttgcttcaggaaatatgactacgtccggcgttccatttggcatgacactacgaccaacacgatctcggttgtctcggcgcactccgtacagtagggatcgcctacctccttttgagacagagacccgcgctaccatactggaggatcatccgctgctgcccgaatgtaacactttgacaatgcacaacgtgagttacgtgcgaggtcttccctgcagtgtgggatttacgctgattcaggaatgggttgttccctgggatatggttctgacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgcatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgtctcatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtaggcgtggacttccccttcgccgcccgttgagcaaccgcaagttggacagcagcctgtggctcagcagctggacagcgacatgaacttaagcgagctgcccggggagtttattaatatcactgatgagcgtttggctcgacaggaaaccgtgtggaatataacacctaagaatatgtctgttacccatgatatgatgctttttaaggccagccggggagaaaggactgtgtactctgtgtgttgggagggaggtggcaggttgaatactagggttctgtgagtttgattaaggtacggtgatcaatataagctatgtggtggtggggctatactactgaatgaaaaatgacttgaaattttctgcaattgaaaaataaacacgttgaaacataacatgcaacaggttcacgattctttattcctgggcaatgtaggagaaggtgtaagagttggtagcaaaagtttcagtggtgtattttccactttcccaggaccatgtaaaagacatagagtaagtgcttacctcgctagtttctgtggattcactagaa

SEQ ID NO: 8 - DNA, 743 bp, Human mastadenovirus C
  source 1..743: mol_type = genomic DNA
cgtaatccgtagatgtacctggacatccaggtgatgccggcggcggtggtggaggcgcgcggaaagtcgcggacgcggttccagatgttgcgcagcggcaaaaagtgctccatggtcgggacgctctggccggtgaggcgtgcgcagtcgttgacgctctagaccgtgcaaaaggagagcctgtaagcgggcactcttccgtggtctggtggataaattcgcaagggtatcatggcggacgaccggggttcgaaccccggatccggccgtccgccgtgatccatgcggttaccgcccgcgtgtcgaacccaggtgtgcgacgtcagacaacgggggagcgctccttttggcttccttccaggcgcggcggctgctgcgctagcttttttggccactggccgcgcgcggcgtaagcggttaggctggaaagcgaaagcattaagtggctcgctccctgtagccggagggttattttccaagggttgagtcgcaggacccccggttcgagtctcgggccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctccccttctcctaccgcgtcaggaggggcaacatcctacatcga

SEQ ID NO: 9 - DNA, 19 bp, synthetic construct
  source 1..19: mol_type = other DNA
atggctgccgatggttatc

SEQ ID NO: 10 - DNA, 23 bp, synthetic construct
  source 1..23: mol_type = other DNA
acgtaatccgtagatgtacctgg

SEQ ID NO: 11 - DNA, 43 bp, synthetic construct
  source 1..43: mol_type = other DNA
aggtacatctacggattacgtaagccgaattctgcagatatcc

SEQ ID NO: 12 - DNA, 39 bp, synthetic construct
  source 1..39: mol_type = other DNA
gtgacctctaatacaggacctagctcccccgataccgtc

SEQ ID NO: 13 - DNA, 22 bp, synthetic construct
  source 1..22: mol_type = other DNA
aggtcctgtattagaggtcacg

SEQ ID NO: 14 - DNA, 47 bp, synthetic construct
  source 1..47: mol_type = other DNA
accatcggcagccatacctgatttaaatcatttattgttcaaagatg

SEQ ID NO: 15 - DNA, 40 bp, synthetic construct
  source 1..40: mol_type = other DNA
aggtacatctacggattacgttgcaggactagaggtcctg

SEQ ID NO: 16 - DNA, 18 bp, synthetic construct
  source 1..18: mol_type = other DNA
agctcccccgataccgtc

SEQ ID NO: 17 - DNA, 20 bp, synthetic construct
  source 1..20: mol_type = other DNA
cgatcgaggtcgacggtatc

SEQ ID NO: 18 - DNA, 23 bp, synthetic construct
  source 1..23: mol_type = other DNA
acgtaatccgtagatgtacctgg

SEQ ID NO: 19 - DNA, 35 bp, synthetic construct
  source 1..35: mol_type = other DNA
ccccctcgatcgaggatgccggggttttacgagat

SEQ ID NO: 20 - DNA, 20 bp, synthetic construct
  source 1..20: mol_type = other DNA
cctcccaccagatcaccatc

SEQ ID NO: 21 - DNA, 41 bp, synthetic construct
  source 1..41: mol_type = other DNA
aggtacatctacggattacgtacctgcaaggaacccctagt

SEQ ID NO: 22 - DNA, 41 bp, synthetic construct
  source 1..41: mol_type = other DNA
gagttgggtaccggatccgttcaactgaaacgaatcaaccg

SEQ ID NO: 23 - DNA, 20 bp, synthetic construct
  source 1..20: mol_type = other DNA
ggatccggtacccaactcca

SEQ ID NO: 24 - DNA, 23 bp, synthetic construct
  source 1..23: mol_type = other DNA
acgtaatccgtagatgtacctgg

SEQ ID NO: 25 - DNA, 41 bp, synthetic construct
  source 1..41: mol_type = other DNA
aggtacatctacggattacgtcgatcgaggtcgacggtatc

SEQ ID NO: 26 - DNA, 38 bp, synthetic construct
  source 1..38: mol_type = other DNA
gagttgggtaccggatccaaaaccctggcgttacccaa

SEQ ID NO: 27 - DNA, 20 bp, synthetic construct
  source 1..20: mol_type = other DNA
ggatccggtacccaactcca

SEQ ID NO: 28 - DNA, 23 bp, synthetic construct
  source 1..23: mol_type = other DNA
acgtaatccgtagatgtacctgg

SEQ ID NO: 29 - DNA, 42 bp, synthetic construct
  source 1..42: mol_type = other DNA
cgaagggcgaattcgtttgcaggactagaggtcctgtattag

SEQ ID NO: 30 - DNA, 22 bp, synthetic construct
  source 1..22: mol_type = other DNA
gggtaaataatcacccgagagt

SEQ ID NO: 31 - DNA, 41 bp, synthetic construct
  source 1..41: mol_type = other DNA
aggtacatctacggattacgtttccagtcgggaaacctgtc

SEQ ID NO: 32 - DNA, 38 bp, synthetic construct
  source 1..38: mol_type = other DNA
gagttgggtaccggatccactttatgcttccggctcgt

SEQ ID NO: 33 - DNA, 20 bp, synthetic construct
  source 1..20: mol_type = other DNA
ggatccggtacccaactcca

SEQ ID NO: 34 - DNA, 23 bp, synthetic construct
  source 1..23: mol_type = other DNA
acgtaatccgtagatgtacctgg

SEQ ID NO: 35 - DNA, 41 bp, synthetic construct; identical to SEQ ID NO: 31
  source 1..41: mol_type = other DNA

SEQ ID NO: 36 - DNA, 38 bp, synthetic construct; identical to SEQ ID NO: 32
  source 1..38: mol_type = other DNA

SEQ ID NO: 37 - DNA, 20 bp, synthetic construct
  source 1..20: mol_type = other DNA
ggatccggtacccaactcca

SEQ ID NO: 38 - DNA, 23 bp, synthetic construct
  source 1..23: mol_type = other DNA
acgtaatccgtagatgtacctgg

SEQ ID NO: 39 - DNA, 20 bp, synthetic construct
  source 1..20: mol_type = other DNA
ggatccggtacccaactcca

SEQ ID NO: 40 - DNA, 18 bp, synthetic construct
  source 1..18: mol_type = other DNA
gtcctgcagcctcagtga

SEQ ID NO: 41 - DNA, 36 bp, synthetic construct
  source 1..36: mol_type = other DNA
actgaggctgcaggacgtggagctccagcttttgtt

SEQ ID NO: 42 - DNA, 23 bp, synthetic construct
  source 1..23: mol_type = other DNA
gttcaactgaaacgaatcaaccg

SEQ ID NO: 43 - DNA, 42 bp, synthetic construct
  source 1..42: mol_type = other DNA
cggttgattcgtttcagttgaactgcaggactagaggtcctg

SEQ ID NO: 44 - DNA, 40 bp, synthetic construct
  source 1..40: mol_type = other DNA
gagttgggtaccggatccggatatctgcagaattcggctt

SEQ ID NO: 45 - DNA, 45 bp, synthetic construct
  source 1..45: mol_type = other DNA
aggtacatctacggattacgtgcaggactagaggtcctgtattag

SEQ ID NO: 46 - DNA, 34 bp, synthetic construct
  source 1..34: mol_type = other DNA
gagttgggtaccggatccgcctcagtgagcgagc

SEQ ID NO: 47 - DNA, 20 bp, synthetic construct
  source 1..20: mol_type = other DNA
ggatccggtacccaactcca

SEQ ID NO: 48 - DNA, 23 bp, synthetic construct
  source 1..23: mol_type = other DNA
acgtaatccgtagatgtacctgg

SEQ ID NO: 49 - DNA, 23 bp, synthetic construct
  source 1..23: mol_type = other DNA
cggttgattcgtttcagttgaac

SEQ ID NO: 50 - DNA, 34 bp, synthetic construct; identical to SEQ ID NO: 46
  source 1..34: mol_type = other DNA

SEQ ID NO: 51 - DNA, 20 bp, synthetic construct
  source 1..20: mol_type = other DNA
ggatccggtacccaactcca

SEQ ID NO: 52 - DNA, 23 bp, synthetic construct
  source 1..23: mol_type = other DNA
gttcaactgaaacgaatcaaccg

SEQ ID NO: 53 - DNA, 23 bp, synthetic construct
  source 1..23: mol_type = other DNA
cggttgattcgtttcagttgaac

SEQ ID NO: 54 - DNA, 38 bp, synthetic construct
  source 1..38: mol_type = other DNA
tggagttgggtaccggatccctgcagaattcggcttgg

SEQ ID NO: 55 - DNA, 20 bp, synthetic construct
  source 1..20: mol_type = other DNA
ggatccggtacccaactcca

SEQ ID NO: 56 - DNA, 23 bp, synthetic construct
  source 1..23: mol_type = other DNA
gttcaactgaaacgaatcaaccg

SEQ ID NO: 57 - DNA, 21 bp, synthetic construct
  source 1..21: mol_type = other DNA
ggaacccctagtgatggagtt

SEQ ID NO: 58 - DNA, 16 bp, synthetic construct
  source 1..16: mol_type = other DNA
cggcctcagtgagcga

SEQ ID NO: 59 - DNA, 7395 bp, synthetic construct
  source 1..7395: mol_type = other DNA
ccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaattgcctgttaatcaataaaccggttgattcgtttcagttgaactttggtctctgcgaagggcgaattcgtttaaacctgcaaggaacccctagtgatggagttggccactccctctctgcgcgctcgctcgctcactgaggctgcaggactagaggtcctgtattagaggtcacgtgagtgttttgcgacattttgcgacaccatgtggtcacgctgggtatttaagcccgagtgagcacgcagggtctccattttgaagcgggaggtttgaacgcgcagccgccaagccgaattctgcagatatccatcacactggcggccgctcgactagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgatcgaggtcgacggtatcgggggagctcgcagggtctccattttgaagcgggaggtttgaacgcgcag

SEQ ID NO: 60 - DNA, 4884 bp, synthetic construct
  source 1..4884: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccattcggtacaattcacgcgtcgacattgattattgactagctctggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggcctagagtcgacgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaatcgaattccgctcgagataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtttatttgtgaaatttgtgatgctattgctttatttgtaaccatctagctttatttgtgaaatttgtgatgctattgctttatttgtaaccattataagctgcaataaacaagttaacaacaacaattgcattcattttatgtttcaggttcagggggagatgtgggaggttttttaaagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt